(19) United States
(12) Patent Application Publication
(10) Pub. No.: US 2011/0118802 A1
(43) Pub. Date: May 19, 2011

(54) NERVE STIMULATION DEVICE

(75) Inventor: Takeo Usui, Tokyo (JP)

(73) Assignee: OLYMPUS CORPORATION, Tokyo (JP)

(21) Appl. No.: 12/885,766

(22) Filed: Sep. 20, 2010

(30) Foreign Application Priority Data
Nov. 13, 2009 (JP) 2009-259913

(51) Int. Cl. A61N 1/36

(52) U.S. Cl. 607/11

(57) ABSTRACT

To provide a nerve stimulation device capable of stably stimulating a vagus nerve while preventing variation in a cardiac rate reducing effect. Adopted is a nerve stimulation device including a cardiac beat detecting part that detects a cardiac beat of a heart by a second electrode, a pulse generating part that generates a pulse for stimulating a vagus nerve to a first electrode, and a controller that controls a timing of generation of a pulse by the pulse generating part in synchronization with a cardiac cycle detected by the cardiac beat detecting part.

### BACKGROUND OF THE INVENTION

1. Field of the Invention

The present invention relates to a nerve stimulation device.

This application is based on Japanese Patent Application No. 2009-259913, the content of which is incorporated herein by reference.

2. Description of Related Art

Conventionally, there is known a cardiac treatment device that stimulates a vagus nerve when a cardiac rate is higher than a predetermined rate, and stimulates a heart instead of stimulating a nerve when the cardiac rate is lower than the predetermined rate (see, Japanese Unexamined Patent Application, Publication No. 2004-173790, for example).

Also known is a cardiac treatment device that detects a tachycardia risk event of a heart and, for example, conducts strong parasympathetic nerve stimulation when a tachycardia risk event of a high level such as ventricular arrhythmia occurs, and conducts weak parasympathetic nerve stimulation when a tachycardia risk event of a low level such as atrial arrhythmia occurs (see, Japanese Unexamined Patent Application, Publication No. 2004-180988, for example).

Japanese Unexamined Patent Application, Publication No. 2004-173790 discloses to stimulate the vagus nerve or the heart depending on the cardiac rate, but lacks the description of a specific timing of stimulating the vagus nerve. As to vagus nerve stimulation, it is not always appropriate to blindly stimulate for high cardiac rates, and stimulation made in an inappropriate timing deteriorates the cardiac rate reducing effect. In other words, if strong stimulation is made in an inappropriate timing, not only the power consumption increases, but also the possibility of damaging a nerve tissue increases. If the timing of stimulating a nerve is variable, the cardiac rate reducing effect may also change depending on the timing even when the cardiac rate is constant.

Japanese Unexamined Patent Application, Publication No. 2004-180988 describes to detect the tachycardia risk event of the heart, and selectively use a single pulse or a burst (triplet) pulse depending on the level of the risk. However, it fails to describe a specific manner of setting the strength of nerve stimulation depending on the cardiac rate. For example, blindly generating continuous burst pulses in response to high cardiac rate may possibly damage a nerve tissue.

Neither Japanese Unexamined Patent Application, Publication No. 2004-173790 nor Japanese Unexamined Patent Application, Publication No. 2004-180988 discloses a timing of stimulating a vagus nerve in one cardiac cycle. However, it is thought that the effect of nerve stimulation on a biological body differs depending on the timing in one cardiac cycle.

### BRIEF SUMMARY OF THE INVENTION

The present invention adopts a nerve stimulation device including a cardiac beat detecting unit that detects a cardiac beat of a heart, a nerve stimulating unit that generates a pulse for stimulating a vagus nerve, and a controlling unit that controls a timing of generating a pulse by the nerve stimulating unit in synchronization with a cardiac cycle detected by the cardiac beat detecting unit.

According to the present invention, the controlling unit controls the timing of generating the pulse for stimulating the vagus nerve by the nerve stimulating unit in synchronization with the cardiac cycle detected by the cardiac beat detecting unit. By synchronizing the timing of stimulation with the cardiac beat in this manner, it is possible to vary the frequency of stimulation according to the increase or decrease in cardiac rate. In other words, when the cardiac rate is high, the frequency of stimulation also increases, so that strong stimulation can be given to the vagus nerve, and the cardiac rate reducing effect can be increased.

On the other hand, when the cardiac rate is low, the frequency of stimulation also decreases, so that weak stimulation can be given to the vagus nerve, and the cardiac rate reducing effect can be decreased. When the cardiac rate is constant, the frequency of stimulation is also constant, so that stimulation of the same level can be given to the vagus nerve in a stable manner, and a stable cardiac rate reducing effect can be obtained.

In the above invention, the controlling unit may make the nerve stimulating unit generate a pulse in a cardiac refractory period.

The term “cardiac refractory period” refers to a period directly after excitation of the cardiac ventricle during which the heart fails to react with any stimulation. Specifically, it corresponds to the period directly after generation of an R wave on an electrocardiogram, and the heart will never be excited even if stimulation is made in this cardiac refractory period. Therefore, by stimulating the vagus nerve in the cardiac refractory period as described above, the possibility that the nerve stimulation signal encircling the heart stimulates the myocardium of the heart to deteriorate the cardiac rate reducing effect is avoided, and stable nerve stimulation is realized.

In the above invention, the controlling unit may stop a pulse by the nerve stimulating unit when the cardiac beat interval detected by the cardiac beat detecting unit is more than or equal to a predetermined threshold.

When the cardiac beat interval is more than or equal to the predetermined threshold, it is not necessary to reduce the cardiac rate by stimulating the vagus nerve because it is a normal pulse or bradycardia condition. Therefore, by stopping stimulation on the vagus nerve when the cardiac beat interval is more than or equal to the predetermined threshold as described above, electric power consumption is decreased, and such a side effect that the cardiac rate is excessively reduced due to too much stimulation, or damage on a nerve tissue can be prevented.

In the above invention, after a lapse of a certain delay time from detection of a standard state of cardiac beat by the cardiac beat detecting unit, the controlling unit may make the nerve stimulation unit generate a pulse.

By generating a pulse after a lapse of a certain delay time from detection of a standard state of cardiac beat (for example, an R wave on an electrocardiogram), for example, it is possible to stimulate a vagus nerve in a cardiac refractory period, and to achieve efficient nerve stimulation. By setting the certain delay time to be shorter than the cardiac cycle detected by the cardiac beat detecting unit, one pulse can be generated in one cardiac cycle, so that a stable cardiac rate reducing effect is obtained.

In the above invention, the controlling unit may make the nerve stimulating unit generate a pulse after a lapse of a delay time that is in proportion to a cardiac cycle from detection of a standard state of cardiac beat by the cardiac beat detecting unit.

By generating a pulse after a lapse of a delay time that is in proportion to a cardiac cycle from detection of the standard state of cardiac beat (for example, an R wave on an electrocardiogram), it is possible to change the timing of stimulation in accordance with the cardiac cycle. As a result, even when the cardiac cycle varies, it is possible to stimulate a vagus nerve, for example, in a cardiac refractory period, and to conduct efficient nerve stimulation.

According to the present invention, an advantage arises that variation in cardiac rate reducing effect is prevented, and the vagus nerve can be stably stimulated.

### BRIEF DESCRIPTION OF THE SEVERAL VIEWS OF THE DRAWINGS

FIG. 1 is an overall configuration view of a nerve stimulation device according to one embodiment of the present invention.

FIG. 2 is a functional block diagram of the nerve stimulation device in FIG. 1.

FIG. 3 is a timing chart describing a pulse generating operation by the pulse generating part shown in FIG. 2.

FIG. 4 is a flowchart showing a processing executed by the controlling unit in a third modified example.

### DETAILED DESCRIPTION OF THE INVENTION

A nerve stimulation device 1 according to one embodiment of the present invention will be described below with reference to the drawings.

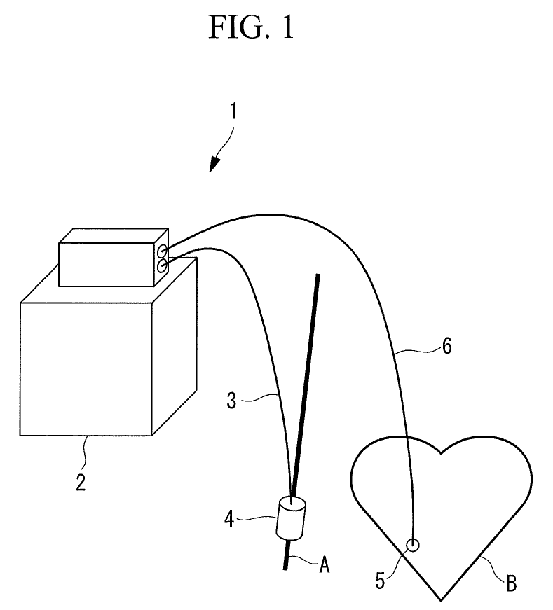

As shown in FIG. 1, the nerve stimulation device 1 according to the present embodiment includes a device main body 2 implanted into a body, a first electrode part 4 attached to a vagus nerve A, a stimulation lead 3 that connects the device main body 2 and the first electrode part 4, a second electrode part 5 attached to heart B, and a cardiac beat detection lead 6 that connects the device main body 2 and the second electrode part 5.

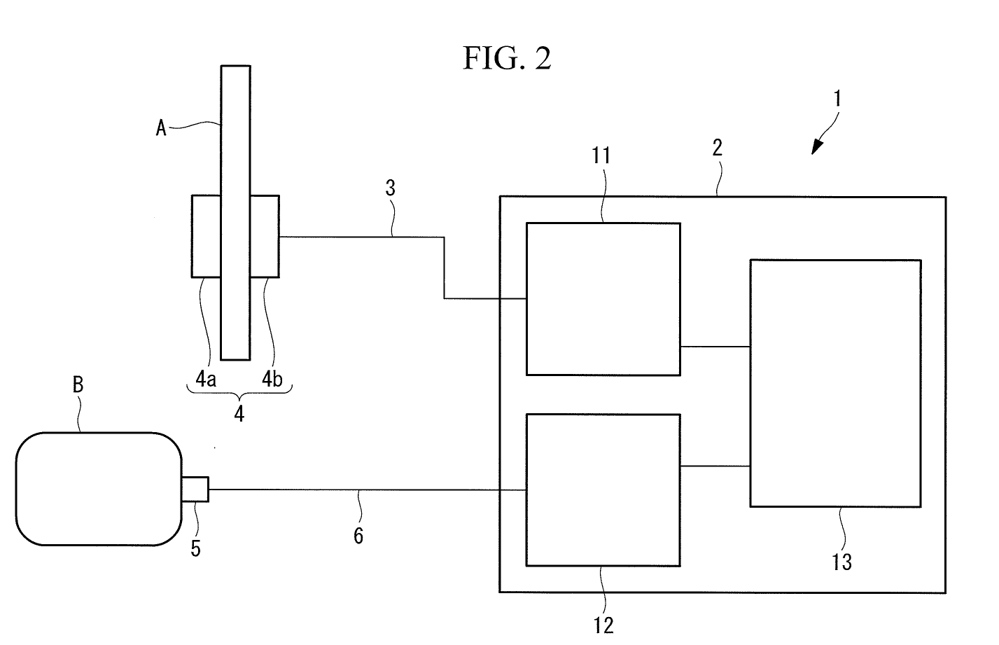

As shown in FIG. 2, the device main body 2 includes a pulse generating part (nerve stimulating unit) 11 that outputs a stimulation pulse to the first electrode part 4, a cardiac beat detecting part (cardiac beat detecting unit) 12 that detects a cardiac beat from an electrocardiographic signal from the heart B detected by the second electrode part 5, and a controller 13 that controls output of a stimulation pulse by the pulse generating part 11.

The first electrode part 4 has an anode electrode 4a and a cathode electrode 4b that are electrically insulated from each other. Each of the electrodes 4a and 4b is formed, for example, into a cylindrical shape, and is adapted to be attached to circumferentially cover the lateral face of the vagus nerve A. The electrodes 4a and 4b are spaced from each other in the longitudinal direction of the vagus nerve A.

The pulse generating part 11 generates a stimulation pulse, and outputs the stimulation pulse to each of the electrodes 4a and 4b via the lead 3. As a result, the vagus nerve A is stimulated by the stimulation pulse in the position situated between the electrodes 4a and 4b, and exited, so that the cardiac rate is decreased. The pulse generating part 11 increases or decreases energy of a stimulation pulse by lengthening or shortening a pulse width of the generated stimulation pulse, thereby enhancing or attenuating the stimulation to be given to the vagus nerve A.

The second electrode part 5 is attached, for example, to the right ventricle of the heart B, and adapted to detect an electrocardiographic signal from the heart B.

The cardiac beat detecting part 12 is adapted to detect a cycle of R wave that peaks on an electrocardiogram and indicates an excited state of the cardiac ventricle from the electrocardiographic signal from the heart B detected by the second electrode part 5.

The controller 13 makes the pulse generating part 11 supply the vagus nerve A with a stimulation pulse, for example, intermittently on a certain cycle. The controller 13 is adapted to control a generation timing of a stimulation pulse by the pulse generating part 11 in synchronization with the cardiac cycle detected by the cardiac beat detecting part 12.

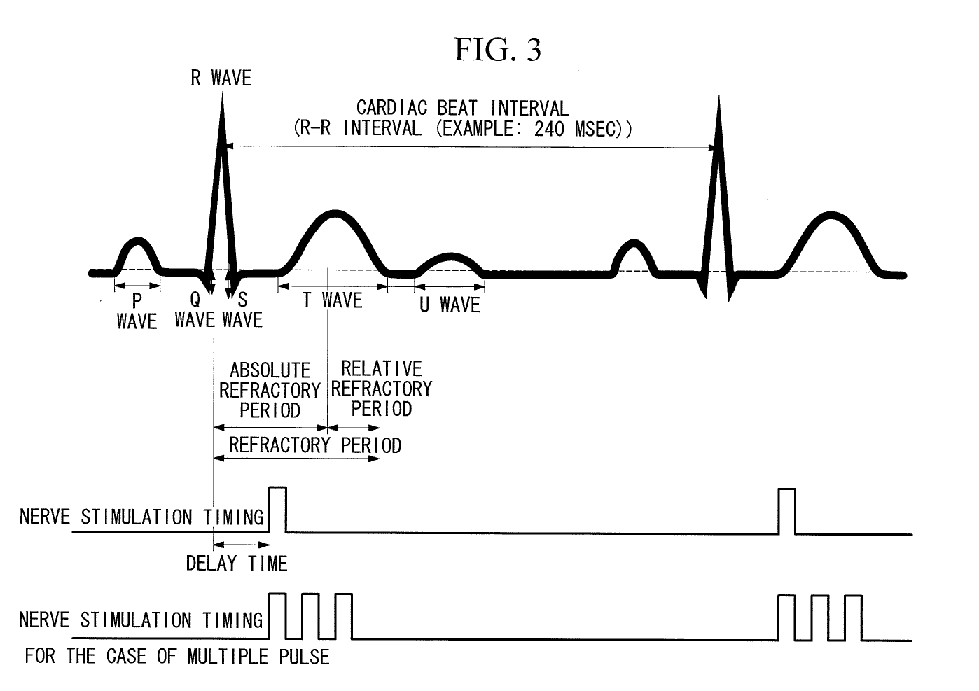

Specifically, as shown in FIG. 3, the controller 13 is adapted to make the pulse generating part 11 generate a pulse after a lapse of a certain delay time from detection of an R wave by the cardiac beat detecting part 12. As to the delay time, for example, when an interval of R waves detected by the cardiac beat detecting part 12, namely a cardiac beat interval is 240 msec, it is set to be shorter than the cardiac beat interval (for example, 200 msec).

An operation of the nerve stimulation device 1 having the above configuration will be described below.

The nerve stimulation device 1 according to the present embodiment is implanted into a body of a patient subject to a cardiac treatment, and the first electrode part 4 is disposed, for example, on the vagus nerve A of a cervical region, and the second electrode part 5 is attached, for example, to the right ventricle of the heart B. Upon start of the operation, the nerve stimulation device 1 stimulates the vagus nerve A intermittently with a stimulation pulse from the pulse generating part 11.

In this case, the controller 13 controls the generation timing of a stimulation pulse by the pulse generating part 11 in synchronization with the cardiac cycle detected by the cardiac beat detecting part 12. Specifically, the controller 13 makes the pulse generating part 11 generate a pulse after a lapse of a delay time (200 msec) shorter than a cardiac beat interval (240 msec) from detection of the R wave by the cardiac beat detecting part 12. In this manner, the pulse generating part 11 necessarily outputs a stimulation pulse once per one cardiac beat, or per one cycle.

Generally, a heart has a certain cycle made up of systole and diastole. Specifically, a heart dilates or contracts by transmission of excitation by electric stimulation to a cardiac conduction system (sinus node→atrioventricular node→His bundle→right/left bundle branch→Purkinje fibers). By conducting nerve stimulation in either timing in this one cycle, and delaying the transmission of excitation, it is possible to delay the cardiac beat. Therefore, by necessarily conducting nerve stimulation in one cycle, it is possible to conduct efficient nerve stimulation without excess and deficiency.

In contrast, when stimulation is conducted randomly irrespective of the cycle, stimulations may be made plural times in one cycle, or no stimulation may be given in one cycle. In such a case, transmission of excitation is delayed, so that not only the cardiac beats cannot be reduced effectively, but also arrhythmia may be caused as a side effect.

On the other hand, according to the nerve stimulation device 1 of the present embodiment, the controller 13 controls the timing of generating the stimulation pulse for stimulating the vagus nerve A by the pulse generating part 11 in synchronization with the cardiac cycle detected by the cardiac beat detecting part 12. By synchronizing the timing of stimulation with the cardiac beat in this manner, it is possible to change the frequency of stimulation depending on the increase or decrease in cardiac rate. That is, when the cardiac rate is high, the frequency of stimulation is also high, so that strong stimulation can be given to the vagus nerve A, and the cardiac rate reducing effect can be increased.

On the other hand, when the cardiac rate is low, the frequency of stimulation is also low, so that weak stimulation can be given to the vagus nerve A, and the cardiac rate reducing effect can be decreased. When the cardiac rate is constant, the frequency of stimulation is also constant, so that stimulation of the same level can be given to the vagus nerve A stably, and a stable cardiac rate reducing effect can be obtained.

By making the pulse generating part 11 generate a stimulation pulse after a lapse of the certain delay time that is shorter than the cardiac beat interval from detection of an R wave which is the standard state of cardiac beat by the cardiac beat detecting part 12, it is possible to necessarily stimulate the vagus nerve A at least once in one cycle, and to conduct stable nerve stimulation.

A stimulation pulse outputted to the vagus nerve A may be made up of one pulse or made up of a plurality of pulses.

### First Modified Example

As a first modified example of the nerve stimulation device 1 according to the present embodiment, the controller 13 may make the pulse generating part 11 generate a pulse in a refractory period of the heart B.

Specifically, as shown in FIG. 3, the controller 13 detects an R wave by the cardiac beat detecting part 12, and makes the pulse generating part 11 generate a stimulation pulse in a certain time from the R wave.

Here the term “cardiac refractory period” refers to a period directly after excitation of the cardiac ventricle during which the heart fails to react with any stimulation. Specifically, it corresponds to the period directly after generation of an R wave on an electrocardiogram, and the heart will never be excited even if stimulation is made in this cardiac refractory period. Therefore, by stimulating the vagus nerve A in the cardiac refractory period, the possibility that the stimulation pulse encircling the heart B stimulates the myocardium of the heart B to deteriorate the cardiac rate reducing effect is avoided, and stable nerve stimulation is realized.

Since the cardiac refractory period is about 100 msec, a stimulation pulse may be generated by the pulse generating part 11, for example, in about 50 msec, from detection of the R wave rather than directly after detection of the R wave.

### Second Modified Example

As a second modified example of the nerve stimulation device 1 according to the present embodiment, the controller 13 may make the pulse generating part 11 generate a pulse after a lapse of a delay time that is proportional to a cardiac cycle from detection of a standard state of cardiac beat by the cardiac beat detecting part 12.

Specifically, as shown in FIG. 3, the controller 13 detects an interval of R waves by the cardiac beat detecting part 12, and sets the delay time, for example, at 100 msec when the interval of R waves is 120 msec, if the delay time at an interval of R waves of 240 msec is set at 200 msec, for example. In this manner, the pulse generating part 11 is able to necessarily output a stimulation pulse once per one cardiac beat, namely per one cycle.

By generating a stimulation pulse after a lapse of a delay time that is in proportion to a cardiac cycle, it is possible to change the timing of stimulation in accordance with the cardiac cycle. As a result, even when the cardiac cycle varies, it is possible to stimulate the vagus nerve A, for example, in the refractory period of the heart B, and to conduct efficient nerve stimulation.

### Third Modified Example

As a third modified example of the nerve stimulation device 1 according to the present embodiment, the controller 13 may make the pulse generating part 11 stop pulsing when a cardiac beat interval detected by the cardiac beat detecting part 12 is more than or equal to a predetermined threshold.

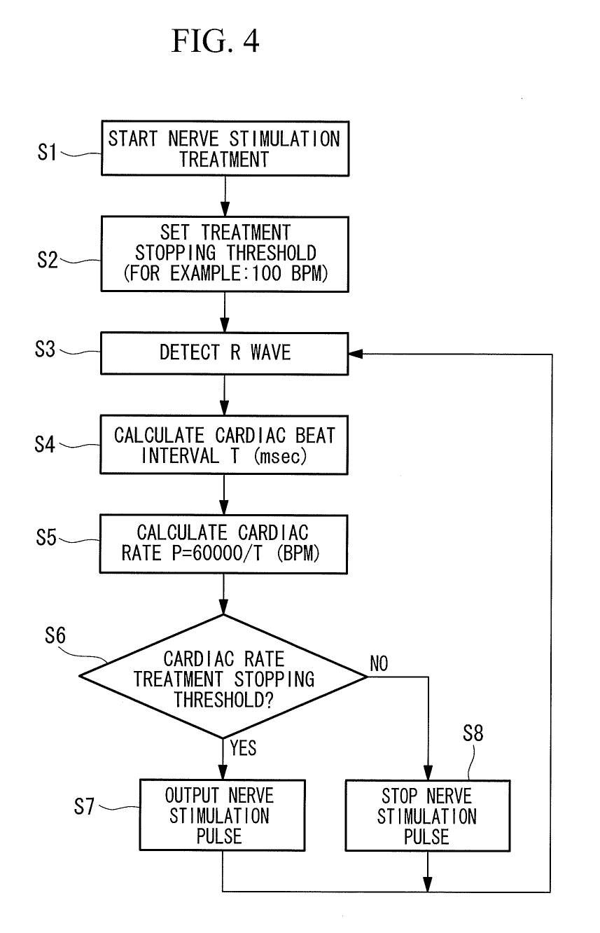

A processing conducted by the controller 13 of the nerve stimulation device 1 according to the present modified example will be described using a flowchart shown in FIG. 4.

First, a nerve stimulation treatment on the vagus nerve A is started (step S1), and a threshold for stopping the nerve stimulation treatment (treatment stopping threshold) is set (step S2). Here, the treatment stopping threshold is set, for example, at 100 beats/minute.

Next, an R wave is detected by the cardiac beat detecting part 12 (step S3), and a cardiac beat interval T is calculated (step S4). Then a cardiac rate P is calculated from the cardiac beat interval T (step S5).

Next, magnitude relation between the cardiac rate P calculated in this manner and the treatment stopping threshold (100 beats/minute) set in step S2 is determined (step S6).

In step S6, when the cardiac rate P is more than or equal to the treatment stopping threshold, a stimulation pulse is outputted, and the nerve stimulation treatment on the vagus nerve A is continued (step S7). On the other hand, when the cardiac rate P is smaller than the treatment stopping threshold, output of the stimulation pulse is stopped, and the treatment of nerve stimulation on the vagus nerve A is stopped (step S8).

When the cardiac rate is less than a predetermined threshold, it is not necessary to reduce the cardiac rate by stimulating the vagus nerve A because it is in a normal pulse or bradycardia condition. Therefore, by stopping stimulation on the vagus nerve A when the cardiac rate P is less than the treatment stopping threshold as in the case of the present modified example, electric power consumption is decreased and such a side effect that the cardiac rate is excessively reduced due to too much stimulation, or damage on a nerve tissue can be prevented.

In the above, embodiments of the present invention have been specifically described with reference to the drawings, however, specific configurations are not limited to these embodiments, and design changes and the like without departing from the scope of the present invention are also included.

For example, the specification of nerve stimulation by the pulse generating part 11 is not limited to the form as described above, and may be appropriately changed depending on the condition of the heart of an individual patient. Specifically, the pulse generating part 11 may increase or decrease energy of a stimulation pulse by lengthening or shortening the pulse width of a stimulation pulse to be generated depending on the condition of the heart of the patient, thereby enhancing or attenuating the stimulation to be given to the vagus nerve A.

Stimulating unit on the vagus nerve is not limited, and it goes without saying that the vagus nerve may be stimulated directly or indirectly (for example, stimulating the vagus nerve indirectly from inside a superior vena cava).

What is claimed is:

1. A nerve stimulation device comprising:
a cardiac beat detecting unit that detects a cardiac beat of a heart;
a nerve stimulating unit that generates a pulse for stimulating a vagus nerve; and
a controlling unit that controls a timing of generating a pulse by the nerve stimulating unit in synchronization with a cardiac cycle detected by the cardiac beat detecting unit.

2. The nerve stimulation device according to claim 1, wherein the controlling unit makes the nerve stimulating unit generate a pulse in a cardiac refractory period.

3. The nerve stimulation device according to claim 1, wherein the controlling unit makes the nerve stimulating unit stop pulsing when a cardiac beat interval detected by the cardiac beat detecting unit is more than or equal to a predetermined threshold.

4. The nerve stimulation device according to claim 1, wherein the controlling unit makes the nerve stimulating unit generate a pulse after a lapse of a certain delay time from detection of a standard state of cardiac beat by the cardiac beat detecting unit.

5. The nerve stimulation device according to claim 1, wherein the controlling unit makes the nerve stimulating unit generate a pulse after a lapse of a delay time that is in proportion to a cardiac cycle from detection of a standard state of cardiac beat by the cardiac beat detecting unit.
